SEQUENCE LISTING (18 sequences)

SEQ ID NO: 1 - DNA, 25 bp, Artificial Sequence
  synthetic oligonucleotide
taacagtcctggtgatatttggtca

SEQ ID NO: 2 - RNA, 25 nt, Artificial Sequence
  Synthetic Oligo
ugaccaaauaucaccaggacuguua

SEQ ID NO: 3 - DNA, 20 bp, Artificial Sequence
  Synthetic Primer
ctagccccgcagacactagc

SEQ ID NO: 4 - DNA, 22 bp, Artificial Sequence
  Synthetic Primer
ccccgcctcctcttgctctgta

SEQ ID NO: 5 - DNA, 22 bp, Artificial Sequence
  Synthetic Primer
tggcgagagagttcctagcctg

SEQ ID NO: 6 - DNA, 21 bp, Artificial Sequence
  Synthetic Primer
tgtgcacaacttcagcccata

SEQ ID NO: 7 - RNA, 27 nt, Artificial Sequence
  Synthetic Oligo
cauggaauccuguuggagcuggucuac

SEQ ID NO: 8 - DNA, 22 bp, Artificial Sequence
  Synthetic Primer
ggctggtgcaggaaagcaacaa

SEQ ID NO: 9 - DNA, 22 bp, Artificial Sequence
  Synthetic Primer
cttgtcaggctagggcgggaag

SEQ ID NO: 10 - DNA, 25 bp, Artificial Sequence
  Synthetic Primer
cccagtctctgcctagatcaaatgg

SEQ ID NO: 11 - DNA, 22 bp, Artificial Sequence
  Synthetic Primer
actcgcacgccttcagtgagaa

SEQ ID NO: 12 - DNA, 22 bp, Artificial Sequence
  Synthetic Primer
atcccagagctgaacgggaagc

SEQ ID NO: 13 - DNA, 22 bp, Artificial Sequence
  Synthetic Primer
ttgggggtaggaacacggaagg

SEQ ID NO: 14 - DNA, 20 bp, Artificial Sequence
  Synthetic Primer
ccgcagctaggaataatgga

SEQ ID NO: 15 - DNA, 20 bp, Artificial Sequence
  Synthetic Primer
ccctcttaatcatggcctca

SEQ ID NO: 16 - RNA, 31 nt, Artificial Sequence
  Synthetic Oligo
gggcaaccguggcuuucgauuguuacugugg

SEQ ID NO: 17 - RNA, 32 nt, Artificial Sequence
  Synthetic Oligo
ggggacaccucggauucuuuuggaucuguggg

SEQ ID NO: 18 - DNA, 25 bp, Artificial Sequence
  Synthetic Oligo
tgtagacaataatgtccatggcctt

(19) United States
(12) Patent Application Publication
(10) Pub. No.: US 2009/0082297 A1
(43) Pub. Date: Mar. 26, 2009

(54) Compositions and Methods for Regulating Gene Expression

(76) Inventors: Daniel T. Lioy, Portland, OR (US); Matthew Klein, Portland, OR (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/146,065

(22) Filed: Jun. 25, 2008

Related U.S. Application Data
(60) Provisional application No. 60/946,081, filed on Jun. 25, 2007.

(51) Int. Cl. A61K 31/7088; C12P 21/04; G01N 33/00; C12Q 1/68; C12N 15/11

(52) U.S. Cl. 514/44; 435/69.1; 436/94; 435/6; 536/23.1

(57) ABSTRACT

Methods, compositions and kits for selectively increasing the expression of a target gene are provided.

This application claims priority under 35 U.S.C. §119(e) to U.S. Provisional Application, 60/946,081 filed Jun. 25, 2007, the entire content of which is incorporated herein by reference.

Pursuant to 35 U.S.C. §202(c), it is acknowledged that the United States Government has certain rights in the invention described herein, which was made in part with funds from the National Institutes of Health Grant No. 1R01NS054742-01A1.

### FIELD OF THE INVENTION

The present invention relates to the fields of medicine, molecular biology and the treatment of disease. More specifically, the invention provides compositions and methods to selectively increase the expression of a target gene.

### BACKGROUND OF THE INVENTION

Several publications are cited throughout the specification in order to describe the state of the art to which this invention pertains. Full citations for these publications are found throughout the specification. Each of these citations is incorporated by reference herein as though set forth in full.

The ability to regulate gene expression in mammals would be advantageous in both experimental and gene therapy settings. In particular, many genes involved in disease processes are repressed at the RNA level.

Classical therapeutics focus on interactions between protein partners in an effort to moderate their disease-intensifying functions. In newer therapeutic approaches, modulation of the actual production of such protein is possible, and by modulating the production of proteins, the maximal therapeutic effect can be obtained with minimal side effects.

RNA interference (RNAi) has become a standard tool to inhibit gene expression, and a variety of nucleic acid species are capable of modifying gene expression. These include antisense RNA, siRNA, microRNA (miRNAs), and RNA and DNA aptamers. Each of these nucleic acid species can inhibit target nucleic acid activity, including gene expression, but a need exists to selectively up-regulate the expression of endogenous genes. Current scientific findings suggest miRNAs may represent a newly discovered, hidden layer of gene regulation.

Much interest has focused on a recently discovered population of non-coding small RNA molecules (i.e., miRNAs) and their effect on intracellular processes, particularly gene expression. MiRNAs are small RNAs, about 15-50 nucleotides in length, which play a role in regulating gene expression in eukaryotic organisms through a naturally occurring process which results in inhibition of expression of the target gene.

Endogenous miRNAs are transcribed as long primary transcripts (pri-miRNA) or embedded in independent non-coding RNAs or in introns of protein-coding genes. Pri-miRNAs are processed into single-stranded mature miRNAs which guide effector complexes, miRNPs, to their target by base-pairing with target mRNAs. MiRNAs are expressed in a wide variety of organisms including worms (nematodes), insects, plants and animals, including humans.

There are many different circumstances where up-regulation of target gene expression is desirable, particularly in certain human diseases. It is an object of the invention to address this need.

### SUMMARY OF THE INVENTION

The present invention is based in part on the discovery that activity of endogenous miRNAs can be inhibited by a miRNA recognition element (MRE)-concealing oligonucleotide, preferably a Locked Nucleic Acid (LNA) oligonucleotide. In accordance with the present invention a method to selectively increase the production of a protein encoded by a target gene of interest is provided. An exemplary method comprises contacting a cell expressing a gene with an oligonucleotide which has binding affinity for a MRE and increasing expression of the target gene of interest. Conditions that may be treated using the methods of the invention include, without limitation, Rett syndrome.

In another embodiment of the invention a method for identifying a LNA oligonucleotide which is effective to up-regulate expression of a target gene of interest is disclosed. An exemplary method entails identifying a MRE in an mRNA encoded by said target gene, and synthesizing a MRE-concealing LNA oligonucleotide, and assessing said LNA oligonucleotide for MRE binding affinity and up-regulation of target gene expression.

In yet another embodiment an isolated oligonucleotide, comprising a nucleotide sequence sufficiently complementary to a (MRE) of about 15-30 nucleotides is provided. In a preferred embodiment the oligonucleotide is conjugated to a chemical moiety to form an antagomir (e.g., cholesterol). This modification facilitates entry into a cell.

In another aspect of the invention, a method of increasing the amount of MeCP2 protein levels in a cell, comprising contacting the cell an MRE-concealing antagomir is disclosed. In a preferred embodiment, the antagomir comprises SEQ ID NO: 1.

Also provided in accordance with the invention are pharmaceutical compositions comprising an antagomir, as described hereinabove, in a pharmaceutically acceptable carrier.

In a further aspect of the invention, kits containing a MRE-concealing LNA oligonucleotide, and appropriate solutions adapted for reconstitution of said oligonucleotide and instructional material are disclosed.

### BRIEF DESCRIPTION OF THE DRAWINGS

FIG. 1. The structure of DNA, RNA, and LNA.

FIG. 2. MicroRNA-132 controls MeCP2 protein levels in P1 cortical neurons. A) addition of miR132, but not miR1-1 or GFP, decreases MeCP2 after 3 days in vitro (DIV). B, C) antisense (AS) 2′-0-me to miR132, but not a scrambled ribonucleotide, increases MeCP2 protein at 5 DIV and blocks the forskolin-induced decrease in MeCP2 and CtBP.

FIG. 3. SEQ ID NO: 1 corresponds to an LNA-modified oligonucleotide that is perfectly antisense to the mir132 MRE in the 3′ UTR of MeCP2 mRNA. *—signifies LNA-modified nucleic acids, and capitalized, bold lettering signifies nucleic acids that participate in base-pairing with mir132.

FIG. 4. A) AS LNA to the MeCP2 miR132 MRE, but not an LNA AS to a non-MRE sequence (control LNA), increases MeCP2 but not CtBP protein levels at 5 DIV, and B) AS LNA, but not control LNA, blocks the decrease in MeCP2 protein levels induced by forskolin (untreated represents untransfected cells). C) Introduction of an AS or control LNA into rat cortical neurons 5 DIV does not cause a change in the level of MeCP2 mRNA compared with untransfected cells. The y-axis represents the fold-increase relative to 18S RNA levels.

FIG. 5. MeCP2 overexpression induces of MeCP2 target genes. The y-axis of figures A-C represents fold increase relative to 18S RNA. A) RT-PCR analysis of target gene expression in cortical cultures 3 DIV following expression of MeCP2 or GFP, B) target gene expression after introduction of 2′-0-me oligo AS to miR132 or a scrambled oligo (Scram), C) Target gene expression after introduction of siRNA to MeCP2 (inset, Western blot of MeCP2 protein levels) and AS 2′-0-me. Control is a scrambled siRNA. All reactions were normalized to 18s levels. Asterisk denotes significant changes (p<0.001).

FIG. 6. Model for homeostatic regulation of MeCP2 mRNA by miR-132.

FIG. 7. A) Primary rat cortical neurons treated with 1 μM MRE-concealing LNA antagomir for 5 days. B) Model for increasing MeCP2 mRNA (Compare with FIG. 6).

### DETAILED DESCRIPTION OF THE INVENTION

The ability to treat diseases associated with decreased gene expression should be premised on the biology underlying protein transcription and translation. If decreased expression of a gene is linked to the development of disease, the compositions and methods described herein can be used to facilitate an increase in the expression of endogenous genes to provide a means for treating an array of disease states. This invention provides compositions and methods to directly increase the expression of a target gene of interest with the ability to differentiate the activities of miRNAs on distinct genes. Accordingly, many diseases and disorders that arise from suboptimal translation of mRNA benefit from the invention.

MiRNAs are a class of 15-30 nt non-coding RNAs (ncRNAs) that exist in a variety of organisms, and are conserved throughout evolution. MiRNAs are processed from hairpin precursors of 70 nt (pre-miRNA) which are derived from primary transcripts (pri-miRNA) through sequential cleavage by RNAse III enzymes. Many miRNAs can be encoded in intergenic regions, hosted within introns of pre-mRNAs or within ncRNA genes. MiRNAs also tend to be clustered and transcribed as polycistrons and often have similar spatial temporal expression patterns. MiRNAs have been found to have roles in a variety of biological processes including developmental timing, differentiation, apoptosis, cell proliferation, organ development, and metabolism. MiRNAs negatively regulate gene expression by incompletely base-pairing to a target sequence in an mRNA. Among the earliest miRNAs genes to be discovered, lin-4 and let-7, base-pair incompletely to repeated elements in the 3′ untranslated regions (UTRs) of other heterochronic genes, and regulate the translation directly and negatively by antisense RNA-RNA interaction (Lee et al. (1993), Cell 75:843-854; Reinhart et al. (2000), Nature 403: 901-906). Several miRNAs and siRNAs function by base-pairing with MREs found in their mRNA targets and direct either target RNA endonucleolytic cleavage (Elbashir et al. (2001); Hutvagner and Zamore (2002)) or translational repression (Olsen and Ambros (1999); Seggerson et al. (2002); Zeng et al. (2002); Doench et al. (2003)).

The recognition region in the mRNA transcript is a miRNA recognition element (MRE), and also referred to in the art as a miRNA response element. MREs are often degenerate sequences, and one miRNA can recognize MREs on more than one gene. This allows the ability to design high-affinity, high-specificity compounds that can distinguish between MREs of similar sequence.

In a particular embodiment, the oligonucleotides of the instant invention comprise modified bases such that the oligonucleotides retain their ability to bind other nucleic acid sequences, but are unable to associate significantly with proteins such as the miRNA degradation machinery. In a preferred embodiment, the invention contemplates the use of previously described oligonucleotides known as “Locked Nucleic Acids” (LNAs), as described in WO 99/14226 and U.S. Pat. No. 6,268,490. LNAs are not required, and are a preferred embodiment within the scope of the invention. In accordance with the present invention the means to identify effective LNAs for use in the methods to increase gene expression are disclosed. For increased nuclease resistance and/or binding affinity to the target, the oligonucleotide agents featured in the invention can also include 2′-O-methyl, 2′-fluorine, 2′-O-methoxyethyl, 2′-O-aminopropyl, 2′-amino, and/or phosphorothioate linkages and the like, as disclosed in Uhlmann et al., Chemical Review, 90: 544-584 (1990). Inclusion of LNAs, ethylene nucleic acids (ENAS), e.g., 2′-4′-ethylene-bridged nucleic acids, and certain nucleobase modifications such as 2-amino-A, 2-thio (e.g., 2-thio-U), G-clamp modifications, can also increase binding affinity to the target.

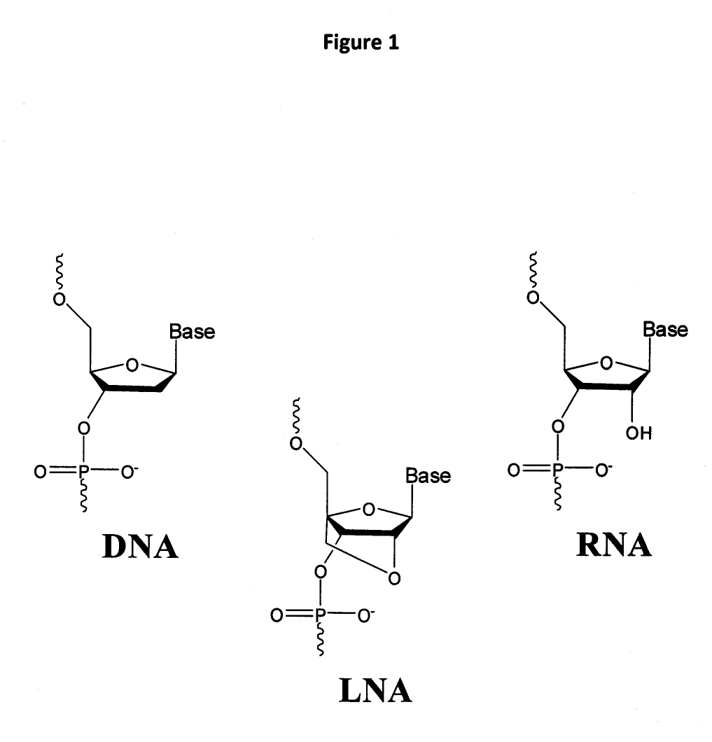

The preferred LNA modification of DNA consists of the addition of a methylene bridge which connects the 2′-O (oxygen) to the 4′-C (carbon) in the ribose ring of a nucleic acid (FIG. 1). This modification provides valuable improvements to oligonucleotides in regard to affinity and specificity to complementary DNA and RNA oligomers, and greatly increases the stability of DNA oligonucleotides in vitro and in vitro because the oligonucleotide is resistant to enzyme degradation. In addition, the LNA modification increases the affinity for target gene sequences. LNA oligonucleotides are highly selective for antisense sequences, and also lack toxicity. The compositions contemplated throughout this application are synthetic, stable, single-stranded oligonucleotides. In particular, LNA-modified oligonucleotides are also referred to herein as “MRE-concealing LNAs” which are also technically “DNA-LNA mixmers.”

In another embodiment of the invention, the MRE-concealing LNAs can be made cell permeable through conjugation to a cholesterol moiety, also known to the skilled artisan as an antagomir. This addition to the LNA oligonucleotide eliminates the need for transfection or other vector delivery methods while improving stability and distribution. The oligonucleotide antagomir can further be in isolated form or can be part of a pharmaceutical composition used for the methods described herein, particularly as a pharmaceutical composition formulated for parental administration. The pharmaceutical compositions can contain one or more oligonucleotide agents, and in some embodiments, will contain two or more oligonucleotide agents, each one directed to a different miRNA.

An antagomir that is substantially complementary to a nucleotide sequence of an MRE can be delivered to a cell or a human to reduce the activity of an endogenous miRNA (e.g., miRNA of an endogenous gene) by creating a competition for binding to a MRE on an mRNA. This is particularly useful in cases when sufficient translation of a target mRNA is blocked by the miRNA. In one embodiment, an antagomir featured in the invention has a nucleotide sequence that is substantially homologous to miR-132, which hybridizes to several RNAs.

The MRE-concealing LNAs is designed as antisense to a specific MRE which allows for selective up-regulation of a target mRNA. In this way, other mRNA-miRNA interactions can still occur in the cell which would target the transcript for degradation by RNase. The oligonucleotides occupy the MRE which prevents miRNA mediated-degradation of the mRNA transcript prior to translation.

An mRNA transcribed from the target gene hybridizes to a miRNA, which consequently results in down-regulation of mRNA expression. An antagomir featured in the invention hybridizes to the MRE which results in an increase in mRNA expression. In the case of a whole organism, the method can be used to increase expression of a gene and treat a condition associated with a low level of expression of a gene. Accordingly this method allows for the blocking of miRNA activity on a single target gene, and given that miRNAs regulate the expression of the majority of genes, this invention has broad applications for therapy.

The invention is based on evidence that indicates inhibiting the ability of microRNA to interact with RNA transcripts would allow for increased translation of the gene product. The MRE-concealing LNAs of the invention can be modified at specific nucleic acids. In the preferred embodiment, the MRE-concealing LNA is actually a “LNA/DNA mixmer” in which only specific nucleic acids are modified. Previous studies have demonstrated that a span of 6 or less unmodified bases prevent RNase H activity which can degrade RNA-DNA duplexes. The MRE-concealing LNAs of the invention contain no more than six unmodified bases in a row. To maximize the stability of the LNAs, greater than 20% of the bases should be modified. When designing MRE-concealing LNA molecules, consideration should also be given to which bases of the MRE in the mRNA that facilitate binding with a miRNA. This step allows the LNA oligo to compete with the miRNA for the target mRNA binding site. Also, to prevent the formation of secondary structures in the LNA oligos, positions should be modified to decrease the thermodynamic stabilities of major secondary structures. Most preferably, one would want to decrease the stability of possible secondary structures that could form at body temperature (37° C.).

In a preferred embodiment, the tag or conjugate is a lipophilic moiety, e.g., cholesterol, which enhances entry of the antagomir into a cell; for example, a hepatocyte, synoviocyte, myocyte, keratinocyte, leukocyte, endothelial cell (e.g., a kidney cell), B-cell, T-cell, epithelial cell, mesodermal cell, myeloid cell, neural cell, neoplastic cell, mast cell, or fibroblast cell. In some embodiments, a myocyte is a smooth muscle cell or a cardiac myocyte. A fibroblast cell can be a dermal fibroblast, and a leukocyte can be a monocyte. In another embodiment, the cell is from an adherent tumor cell line derived from a tissue, such as bladder, lung, breast, cervix, colon, pancreas, prostate, kidney, liver, skin, or nervous system (e.g., central nervous system).

The invention also contemplates using the MRE-concealing oligonucleotides of the invention expressed from transcriptional units inserted into nucleic acid vectors. The recombinant vectors can be DNA plasmids or viral vectors. Oligonucleotide-expressing viral vectors can be constructed based on, but not limited to, adeno-associated virus, retrovirus, adenovirus, or alphavirus. The recombinant vectors capable of expressing the oligonucleotide agents can be delivered as described herein, and can persist in target cells. Alternatively, viral vectors can be used that provide for transient expression of nucleic acid molecules.

MRE-concealing LNAs may include variants which are at least about 75%, 80%, 85%, 90%, or 95%, and often, more than 90%, or more than 95% homologous to the MRE sequence in the 3′ UTR of a transcript. The prediction of miRNA targets is well known in the art and can be found, for example, on the world wide web at (mami.med.harvard.edu). All homology may be computed by algorithms known in the art, such as BLAST, described in Altschul et al., J. Mol. Biol. (1990) 215:403-10). In addition, the sequences may comprise a nucleotide sequence which results from the addition, deletion or substitution of at least one nucleotide to the 5′-end and/or the 3′-end of one or more of oligonucleotides, or a derivative thereof.

One can evaluate a candidate single-stranded oligonucleotide agent for a selected property by exposing the agent or modified molecule and a control molecule to the appropriate conditions and evaluating for the presence of the selected property. For example, resistance to a degradation agent can be evaluated by exposure, for example, to a nuclease, or a biological sample likely encountered during therapeutic use, such as blood. A parameter, for example, size, can be determined by a method known to those skilled in the art to assess whether the molecule has maintained its original length, or functionality. In this regard, functional assays can be used to evaluate the candidate molecule to determine if there is any alteration in the ability of the molecule to increase gene expression.

Also disclosed is a method for identifying an oligonucleotide which is effective to up-regulate expression of a target gene of interest which entails identifying a MRE in an mRNA encoded by a target gene of interest, synthesizing a MRE-concealing oligonucleotide, and assessing said oligonucleotide for MRE binding affinity and up-regulation of target gene expression.

In addition, the aspects of the invention described above allow for the development of screening methods and small molecule inhibitors of the miRNA-mRNA transcript interaction useful for new therapies are also within the scope of the invention.

The following definitions are provided to facilitate and understanding of the present invention.

### I. DEFINITIONS

The following definitions are provided to facilitate an understanding of the present invention:

As used herein, the terms “nucleic acid”, “polynucleotide” and “oligonucleotide” refer to any DNA, RNA, LNA, primers, probes, oligomer fragments, oligomer controls and blocking oligomers. There is no intended distinction in length between the term “nucleic acid”, “polynucleotide” and “oligonucleotide”, and these terms will be used interchangeably. These terms refer only to the primary structure of the molecule. Thus, these terms include double- and single-stranded DNA, as well as double- and single stranded RNA or a mixture thereof (e.g., LNA-DNA mixmers). The oligonucleotide is comprised of a sequence of approximately at least 3 nucleotides, preferably at least about 6 nucleotides, and more preferably at least about 10-30 nucleotides corresponding to a region of the designated target sequence.

Oligos can be composed of naturally occurring nucleobases, sugars and internucleoside (backbone) linkages as well as oligonucleotides having non-naturally-occurring portions which function similarly or with specific improved functions. Fully or partly modified or substituted oligonucleotides are often preferred over native forms because of several desirable properties of such oligonucleotides, for instance, the ability to penetrate a cell membrane, good resistance to extra- and intracellular nucleases, high affinity and specificity for the nucleic acid target. The LNA oligonucleotides of the invention exhibit the above-mentioned properties.

“Corresponding” means identical to or complementary to the designated sequence. The oligonucleotide is not necessarily physically derived from any existing or natural sequence but may be generated in any manner, including chemical synthesis, DNA replication, reverse transcription or a combination thereof.

Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations on DNA, RNA and other natural and synthetic nucleotides, such as LNAs, also defined herein.

In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a RNA molecule, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The term “Locked Nucleic Acid” (LNA) refers to certain types of modified oligonucleotides which can be prepared as described in International Patent Applications WO 99/14226 and WO 98/39352. Thus, the LNA oligonucleotides may be produced using the oligomerization techniques of nucleic acid chemistry well-known to a person of skill in the art of organic chemistry. LNA oligonucleotides of the invention are useful for a number of therapeutic applications as indicated herein. In general, therapeutic methods of the invention include administration of a therapeutically effective amount of an LNA-modified oligonucleotide to a mammal, particularly a human. The present invention also relates to an LNA oligonucleotide as defined herein or a conjugate as defined herein for use as a medicament.

An “antagomir” as used herein is a chemically modified MRE-concealing LNA oligonucleotide which is conjugated to a chemical moiety to confer a function. Preferably, the antagomir is a LNA oligonucleotide molecule conjugated to lipophilic moiety on the 3′ end of the molecule, such as cholesterol.

The terms “miRNA” and “microRNA” refer to about 10-35 nt, preferably about 15-30 nt, and more preferably about 20-26 nt, non-coding RNAs derived from endogenous genes encoded in the genomes of plants and animals. They are processed from longer hairpin-like precursors termed pre-miRNAs that are often hundreds of nucleotides in length. MicroRNAs assemble in complexes termed miRNPs and recognize their targets by antisense complementarity. These highly conserved, endogenously expressed RNAs are believed to regulate the expression of genes by binding to the 3′-untranslated regions (3′-UTR) of specific mRNAs. Without being bound by theory, a possible mechanism of action assumes that if the microRNAs match 100% their target, i.e. the complementarity is complete, the target mRNA is cleaved, and the miRNA acts like a siRNA. However, if the match is incomplete, i.e. the complementarity is partial, then the translation of the target mRNA is blocked. The manner by which a miRNA base-pairs with its mRNA target correlates with its function: if the complementarity between a mRNA and its target is extensive, the RNA target is cleaved; if the complementarity is partial, the stability of the target mRNA in not affected but its translation is repressed.

“LNA/DNA mixmer” oligonucleotides (i.e., oligonucleotides containing both LNA and DNA nucleotides) is used to refer to a nucleic acid that contains at least one LNA unit and at least one RNA or DNA unit (e.g., a naturally-occurring RNA or DNA unit).

The present invention also includes active portions, fragments, derivatives and functional or non-functional mimetics of the polypeptides of the invention. “Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

The term “functional” as used herein implies that the nucleic or amino acid sequence is functional for the recited assay or purpose.

The term “conjugate” or “tag” refers to a chemical moiety, either a nucleotide, oligonucleotide, polynucleotide or an amino acid, peptide or protein or other chemical, that when added to another sequence, provides additional utility or confers useful properties, particularly in the delivery, trafficking, detection or isolation of that sequence. Preferably, the conjugate is cholesterol added to the 3′ end of the MRE-concealing LNA, which confers the ability of the LNA of the invention to be cell permeable. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Numerous other tag moieties are known to, and can be envisioned by, the skilled artisan, and are contemplated to be within the scope of this definition.

The term “gene” generally refers to a nucleic acid sequence that comprises coding sequences necessary for the production of a nucleic acid (e.g., miRNA or antisense nucleic acid) or a polypeptide (protein) or protein precursor. In addition to the coding sequence, the term gene may also include, in proper contexts, the sequences located adjacent to the coding region on both the 5′ and 3′ ends which correspond to the full-length mRNA (the transcribed sequence) or all the sequences that make up the coding sequence, transcribed sequence and regulatory sequences. The sequences that are located 5′ of the coding region and which are present on the mRNA are referred to as 5′ untranslated region (5′ UTR). The sequences that are located 3′ or downstream of the coding region and which are present on the mRNA are referred to as 3′ untranslated region (3′ UTR). Gene expression can be regulated at many stages in the process. Up-regulation or activation refers to regulation that increases the production of gene expression products (i.e., RNA or protein).

As used herein, “pharmaceutical formulations” include formulations for human and veterinary use with no significant adverse toxicological effect. “Pharmaceutically acceptable formulation” as used herein refers to a composition or formulation that allows for the effective distribution of the nucleic acid molecules of the instant invention in the physical location most suitable for their desired activity. The term “pharmaceutically acceptable carrier” means that the carrier can be taken into the subject with no significant adverse toxicological effects on the subject. The term “therapeutically effective amount” is the amount present that is delivered to a subject to provide the desired physiological response. Methods for preparing pharmaceutical compositions are within the skill in the art, for example as described in Remington's Pharmaceutical Science, 18th ed., Mack Publishing Company, Easton, Pa. (1990), and The Science and Practice of Pharmacy, 2003, Gennaro et al.

The term “co-administration” refers to administering to a subject two or more oligonucleotide agents. The agents can be contained in a single pharmaceutical composition and be administered at the same time, or the agents can be contained in separate formulation and administered serially to a subject. So long as the two agents can be detected in the subject at the same time, the two agents are said to be co-administered.

As used herein, an “instructional material” includes a publication, a recording, a diagram, or any other medium of expression which can be used to communicate the usefulness of the composition of the invention for performing a method of the invention. The instructional material of a kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

### II. FORMULATIONS AND DELIVERY OF SINGLE-STRANDED MRE-CONCEALING LNA OLIGONUCLEOTIDE AGENTS

The single-stranded oligonucleotide agents described herein can be formulated for administration to a subject. It should be understood that these formulations, compositions, and methods can be practiced with both modified (e.g., LNAs and antagomirs) and unmodified oligonucleotide agents within the scope of the invention. Also, a formulated antagomir composition can assume a variety of states.

The compositions (e.g., crystalline, anhydrous or aqueous phase) can be incorporated into a delivery vehicle, e.g., a liposome (particularly for the aqueous phase), or a particle (e.g., a microparticle for a crystalline composition). Generally, the antagomir composition is formulated in a manner that is compatible with the intended method of administration.

An antagomir preparation of the MRE-concealing LNAs can be formulated in combination with another agent, e.g., another therapeutic agent or an agent that stabilizes an oligonucleotide agent, e.g., a protein which complexes with the oligonucleotide agent. Still other agents include, without limitation, chelators, salts, and RNAse inhibitors (e.g., RNAsin).

In one embodiment, the antagomir preparation includes another antagomir, e.g., a second antagomir that can modulate the expression of a second gene. Still other preparations can include at least three, five, ten, twenty, fifty, or a hundred or more different oligonucleotide species. In some embodiments, the agents are directed to the same target nucleic acid but different target sequences. In another embodiment, each antagomir is directed to a different target.

A composition that includes an LNA antagomir featured in the invention, e.g., an antagomir that targets a MRE can be delivered to a subject by a variety of routes depending upon whether local or systemic treatment is desired and upon the area to be treated. Exemplary routes include inhalation, intrathecal, parenchymal, intravenous, nasal, subcutaneous, intraperitoneal, intramuscular, oral, and ocular delivery. In general, delivery of LNA oligo featured in the invention directs the agent to the desired site in a subject. The preferred means of delivery is through local administration directly to the site of infection, or by systemic administration, e.g. parental administration.

An antagomir can be incorporated into pharmaceutical compositions suitable for administration. For example, compositions can include one or more oligonucleotide agents and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

Formulations for direct injection and parenteral administration are well known in the art. Such formulations may include sterile aqueous solutions which may also contain buffers, diluents and other suitable additives. For intravenous use, the total concentration of solutes should be controlled to render the preparation isotonic.

A patient that has been diagnosed with a disease or disorder characterized by reduced gene expression (i.e., under-expression) can be treated by administration of an antagomir described herein to block the binding and effects of an miRNA. This will alleviate the symptoms associated with the disease or disorder.

The oligonucleotide agents featured in the invention can include a delivery vehicle, such as liposomes, for administration to a subject, carriers and diluents and their salts, and/or can be present in pharmaceutically acceptable formulations. Methods for the delivery of nucleic acid molecules are well known in the art.

An antagomir featured in the invention may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained release composition may be appropriate.

An antagomir featured in the invention can be administered in a single dose or in multiple doses. Where the administration of the antagomir is by infusion, the infusion can be a single sustained dose or can be delivered by multiple infusions. Injection of the agent can be directly into the tissue at or near the site of aberrant target gene expression. Multiple injections of the agent can be made into the tissue at or near the site.

Dosage levels on the order of about 1 μg/kg to 100 mg/kg of body weight per administration are useful in the treatment of a disease. In regard to dosage, an antagomir can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of antagomir per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of antagomir per kg of bodyweight. The unit dose, for example, can be administered by injection (e.g., intravenous or intramuscular, intrathecally, or directly into an organ), inhalation, or a topical application.

One skilled in the art can also readily determine an appropriate dosage regimen for administering the antagomir of the invention to a given subject. For example, the antagomir can be administered to the subject once, e.g., as a single injection or deposition at or near the site on unwanted target nucleic acid expression. Alternatively, the antagomir can be administered once or twice daily to a subject for a period of from about three to about twenty-eight days, more preferably from about seven to about ten days. In a preferred dosage regimen, the antagomir is injected at or near a site of repressed target nucleic acid expression once a day for seven days. Where a dosage regimen comprises multiple administrations, it is understood that the effective amount of antagomir administered to the subject can include the total amount of antagomir administered over the entire dosage regimen. One skilled in the art will appreciate that the exact individual dosages may be adjusted somewhat depending on a variety of factors, including the specific antagomir being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. For instance, oral administration generally would be expected to require higher dosage levels than administration by intravenous or intravitreal injection. Variations in these dosage levels can be adjusted using standard empirical routines of optimization, which are well-known in the art.

In addition to treating a pre-existing disease or disorder, the LNA oligonucleotides featured in the invention (e.g., single-stranded LNA oligonucleotide antagomirs) can be administered prophylactically in order to prevent or slow the onset of a particular disease or disorder, such as disorders associated with repressed translation of a mRNA.

The present pharmaceutical formulations include an antagomir featured in the invention (e.g., 0.1 to 90% by weight), mixed with a physiologically acceptable carrier medium. Preferred physiologically acceptable carrier media are water, buffered water, normal saline, 0.4% saline, 0.3% glycine, hyaluronic acid and the like.

Pharmaceutical compositions featured in the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers, additions of chelants or calcium chelate complexes, or, optionally, additions of calcium or sodium salts. Pharmaceutical compositions can be packaged for use in liquid form, or can be lyophilized.

The present invention also features compositions prepared for storage or administration that include a pharmaceutically effective amount of the desired oligonucleotides in a pharmaceutically acceptable carrier or diluent. Acceptable carriers or diluents for therapeutic use are well known in the pharmaceutical art.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

Delivery of an antagomir directly to an organ can be at a dosage on the order of about 0.00001 mg to about 3 mg per organ, or preferably about 0.0001-0.001 mg per organ, about 0.03-3.0 mg per organ, about 0.1-3.0 mg per organ or about 0.3-3.0 mg per organ. The dosage can be an amount effective to treat or prevent a disease or disorder.

In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. Because oligonucleotide agent-mediated up-regulation can persist for several days after administering the antagomir composition, in many instances, it is possible to administer the composition with a frequency of less than once per day, or, for some instances, only once for the entire therapeutic regimen.

In one embodiment, a subject is administered an initial dose, and one or more maintenance doses of an antagomir. The maintenance dose or doses are generally lower than the initial dose, e.g., one-half less of the initial dose. The maintenance doses are preferably administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable. Following successful treatment, it may be desirable to have the patient undergo maintenance therapy to prevent the recurrence of the disease state. The concentration of the antagomir composition is an amount sufficient to be effective in treating or preventing a disorder or to regulate a physiological condition in humans. The concentration or amount of antagomir administered will depend on the parameters determined for the agent and the method of administration.

Certain factors may influence the dosage required to effectively treat a subject, including but not limited to the severity of the disease or disorder, previous treatments, the general health and/or age of the subject, and other diseases present. It will also be appreciated that the effective dosage of the antagomir used for treatment may increase or decrease over the course of a particular treatment. Changes in dosage may result and become apparent from the results of diagnostic assays. For example, the subject can be monitored after administering an antagomir composition. Based on information from the monitoring, an additional amount of the antagomir composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

### III. UP-REGULATION OF TUMOR SUPPRESSOR GENES AND OTHER GENES INVOLVED IN DISEASE

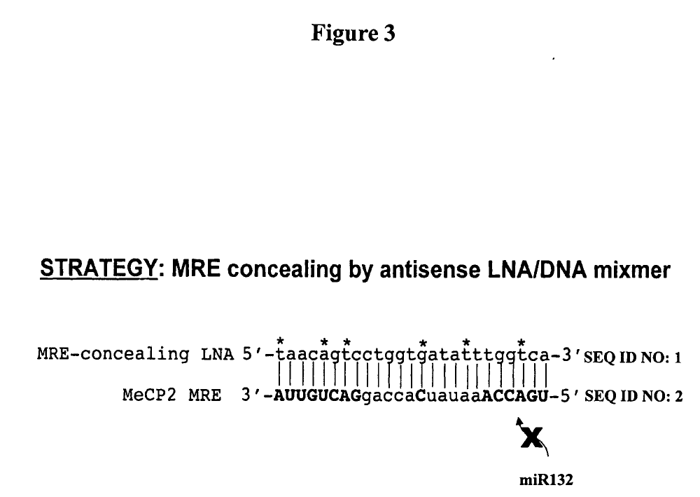

The example provided hereinbelow represents one use of the MRE-concealing LNAs (and antagomirs) of the invention to increase MeCP2 expression. The MRE exemplified FIG. 3 corresponds to the 3′ UTR of MeCP2 from rats; also, the MRE of miR-132 on MeCP2 in rats is also found in mice and humans. It is also within the scope of the invention to increase the expression of other gene products in a variety of disease states. For example, under-expression of tumor suppressor genes contributes to the malignant process. Therefore, the present invention could be used to increase the expression of genes such as, without limitation, p53, p27, RB1, or any other gene in which the protein product is repressed through microRNA-mediated degradation of the mRNA transcript prior to translation. In another context, up-regulation of myotropin would be desirable for the treatment of diabetes. Similarly, the up-regulation of Hand1 can be useful to treat some cardiac disorders.

### IV. IN VIVO TESTING OF MRE-CONCEALING LNAS

To further validate the activity of the oligonucleotides of the invention in vivo, non-human animal models, for example, rat, zebrafish and primate models, can be used. In one aspect of in vivo testing, the oligonucleotides of the invention can be injected into wild type rat brains (e.g., cortex and ventricles), and assessed for an increase in protein (e.g., MeCP2) level corresponding to the MRE target mRNA in the brain. In another aspect, a zebrafish model could be employed. For example, a zebrafish model which has a phenotype associated a mutation in a gene of interest or an under-expression of a gene of interest could be used to screen oligonucleotides of the invention to identify those that can rescue the wild type phenotype of the zebrafish, thereby demonstrating the ability of the oligonucleotide to increase expression of the gene of interest. As another model of in vivo assessment, the antagomirs of the invention could be conjugated with other moieties to facilitate entry into the central nervous system. For example, the oligonucleotides of the invention could be conjugated to a herpesvirus protein which confers neurotropic targeting and facilitates passage of the oligonucleotide across the blood brain barrier.

### V. CLINICAL APPLICATIONS

As mentioned previously, a preferred embodiment of the invention comprises delivery of the MRE-concealing LNA oligonucleotide to a patient in need thereof. Formulation, dosages and treatment schedules have also been described hereinabove. Phase I clinical trials can be designed to assess the safety, tolerability, pharmacokinetics, and pharmacodynamics of the conjugated MRE-concealing LNA oligonucleotides of the invention. These trials may be conducted in an inpatient clinic, where the subject suffering from a disease resulting from low gene expression can be observed by full-time medical staff. After the initial safety of the therapy has been performed, Phase II trails can assess clinical efficacy of the therapy; as well as to continue Phase I assessments in a larger group of volunteers and patients. Subsequently, Phase III studies on large patient groups entails definitive assessment of the efficacy of the MRE-concealing LNA antagomirs for a disease or disorder in comparison with current treatments. Finally, Phase IV trials involving the post-launch safety surveillance and ongoing technical support for the MRE-concealing LNA oligonucleotides could be completed.

### VI. KITS

If the pharmaceutical composition in liquid form is under risk of being subjected to conditions which will compromise the stability of the LNA oligonucleotide, it may be preferred to produce the finished product containing the LNA oligonucleotide in a solid form, e.g. as a freeze dried material, and store the product is such solid form. The product may then be reconstituted (e.g. dissolved or suspended) in a saline or in a buffered saline ready for use prior to administration.

Hence, the present invention also provides a kit comprising (a) a first component containing an LNA oligonucleotide or a conjugate as defined hereinabove in solid form, and (b) a second component containing saline or a buffer solution (e.g. buffered saline) adapted for reconstitution (e.g. dissolution or suspension) of said LNA oligonucleotide.

Preferably said saline or buffered saline has a pH in the range of 4.0-8.5, and a molarity of 20-2000 mM. In a preferred embodiment the saline or buffered saline has a pH of 6.0-8.0 and a molarity of 100-500 mM. In a most preferred embodiment the saline or buffered saline has a pH of 7.0-8.0 and a molarity of 120-250 mM. For such a kit, the LNA oligonucleotide preferably consists of SEQ ID NO. 1.

The following example is provided to illustrate an embodiment of the invention. It is not intended to limit the scope of the invention in any way. It will be appreciated that what follows is by way of example only and that modifications to detail may be made while still falling within the scope of the invention.

### EXAMPLE 1

### MRE-concealing LNAs Inhibit miRNAs and Result in Increased Gene Expression which Benefits a Sub-Population of Patients with Rett Syndrome

Rett syndrome is an Autism spectrum disorder and is a leading cause of mental retardation in females (1). Loss-of-function and hypomorphic mutations in methyl CpG binding protein-2 (MeCP2) cause Rett syndrome. MeCP2 is a sex-linked gene, and therefore, MeCP2 mutations in males are thought to be embryonic lethal.

MeCP2 levels steadily increase from birth to postnatal day 7, a period of prominent synaptic maturation (6). Loss of MeCP2 during this period delays neuronal

maturation and synaptogenesis (13, 14). Both increases and decreases in MeCP2 levels are associated with neurodevelopmental defects. These findings highlight the importance of maintaining a narrow range in MeCP2 levels during neuronal development.

MeCP2 translation is regulated by microRNA 132 (miR132), which recognizes a target within the MeCP2 3′UTR, and also regulates multiple target genes through the recognition of degenerate MREs. The increase in MeCP2 protein caused by inhibition of miR132 function elevates BDNF levels. Blocking the miR132 pathway may

provide an approach for increasing MeCP2 expression in Rett syndrome. Interaction of miR132 with its miRNA recognition element (MRE) in the MeCP2 3′UTR prevents MeCP2 levels from becoming deleteriously high during neuronal maturation.

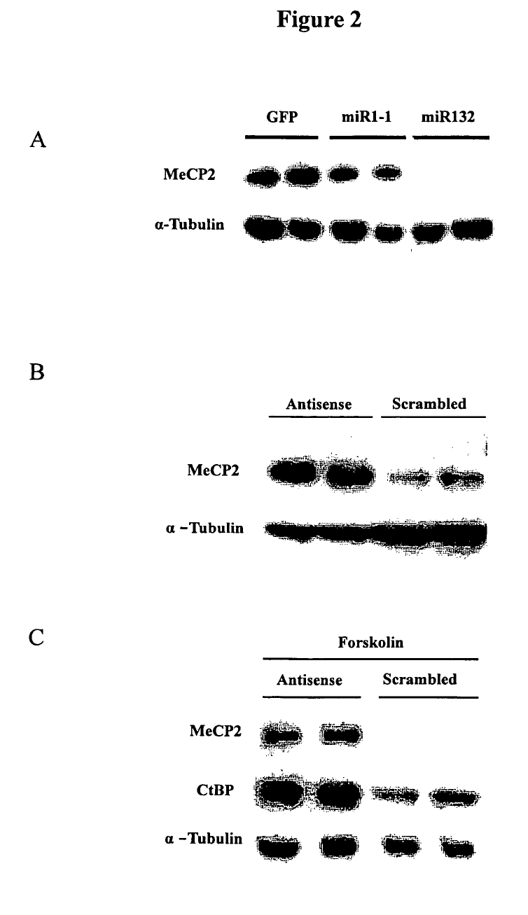

The MeCP2 gene contains multiple polyadenylation sites which result in transcripts with short or long 3′UTRs. The long transcript predominates in brain while the shorter form is expressed in visceral organs and muscle (6). Previously it has been shown that miR132 contributed to BDNF-mediated neurite outgrowth of neonatal neurons (7). Basal levels of miR132 are not appreciable until after birth, suggesting that its principal role may be to regulate proteins involved in later stages of neuronal maturation. Consistent with this idea, introduction of miR132 into primary cortical neurons decreased MeCP2 protein levels (FIG. 2A). This effect was specific. in that miR1-1 (whose MRE is not present in the MeCP2 3′UTR) had no effect.

Conversely, a 2′-0-methyl oligoribonucleotide (2′-0-me) antisense (AS) to miR132 increased MeCP2 protein levels under basal conditions and blocked the decrease induced by forskolin (FIGS. 2B, 2C). The forskolin-induced decrease of C-terminal binding protein (CtBP), another predicted miR132 target, was also blocked after treatment with the AS 2′-0-me blocker.

2′-0-me blockers are believed to inhibit the RNA-induced silencing complex, but in some instances, their effects do not correspond to genetic knockouts (8, 9). To address this problem, locked nucleic acid (LNA) oligonucleotides complementary (i.e., antisense) to the miR132 MRE in the 3′ UTR of MeCP2 were utilized (FIG. 3). The experiments detailed below were performed in a rat model, however the MeCP2 MRE sequence of FIG. 3 is also found in the 3′UTR of mice and humans. Therefore, the MRE-concealing LNA of SEQ ID NO: 1 would be useful in other systems as well.

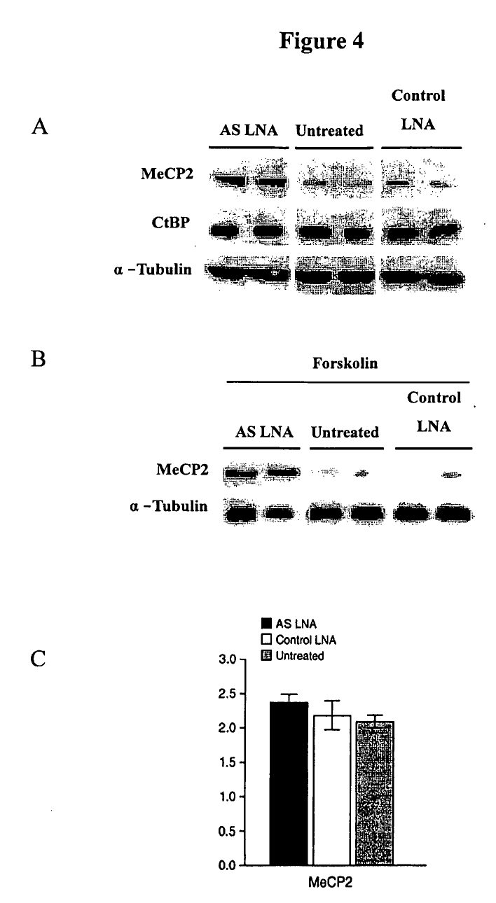

LNAs have a higher affinity for RNA than 2′-0-me blocker (10), and should prevent the binding of microRNAs to their specific 3′UTR targets. A LNA (i.e., a MRE-concealing LNA molecule) designed to block the interaction of miR132 with the MeCP2 MRE increased MeCP2 protein in cortical neurons and blocked the decrease induced by forskolin when rat primary cortical neurons were transfected with the oligo (FIGS. 4A, 4B). No change was seen in MeCP2 mRNA levels (FIG. 4C) or protein levels of CtBP (FIG. 4A). CtBP is another miR132 target harboring a different MRE than MeCP2. This result demonstrates the specificity of the MRE-concealing LNA directed to MeCP2. Furthermore, as demonstrated in FIG. 4A-B, an LNA oligo corresponding to a non-MRE (control LNA) sequence in the MeCP2 3′UTR did not affect MeCP2 levels.

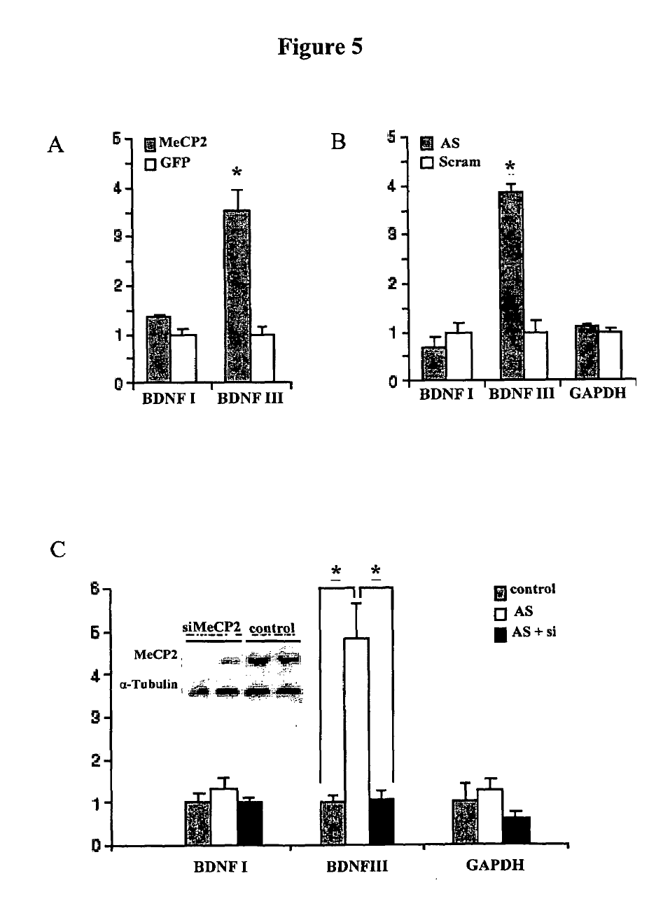

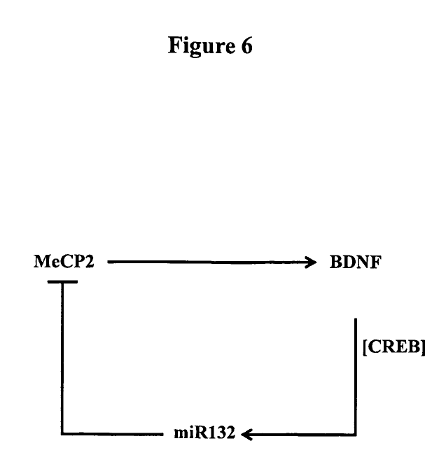

BDNF has received much attention as a MeCP2 target because of its role in neuronal maturation. Mouse models of Rett, which lack MeCP2, have decreased BDNF levels (11), and exogenous BDNF partially rescued their behavioral defects and extended their lifespan (12). MeCP2 elevation effects BDNF expression by increasing BDNF transcript levels. MeCP2 overexpression increased BDNF transcript levels, as did introduction of a 2′-0-me oligo AS to miR132 (FIGS. 5A, 5B). To test whether the BDNF increase induced by the AS 2′-0-me oligo was due to elevated MeCP2 levels, a short hairpin siRNA that reduced levels of MeCP2 protein was utilized (FIG. 5C insert). Co-transfection of the siRNA blocked the effects of the 2′-o-me oligo on BDNF, suggesting that the increase in BDNF was due to MeCP2 and not other miR132 targets (FIG. 5C). The following model exemplifies the homeostatic regulation of MeCP2 (FIG. 6); specifically as MeCP2 levels increase, so does BDNF, which induces miR132 and represses MeCP2 translation.

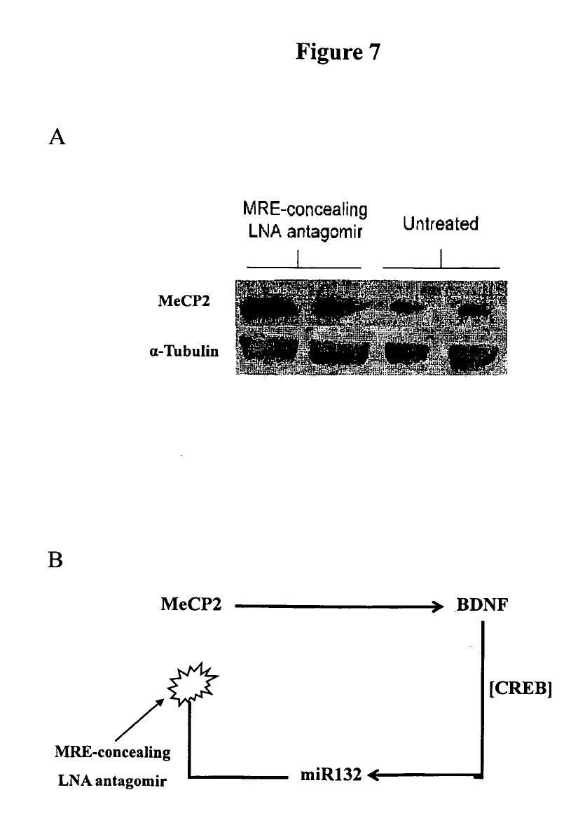

Expression of MeCP2 protein in animal models of Rett syndrome suggests that Rett syndrome is reversible. MRE-concealing LNAs, as demonstrated hereinabove, are valuable treatment molecules for increasing MeCP2 levels. To develop an MRE-concealing LNA oligo as a therapeutic, the MRE-concealing LNA described in FIG. 3, consisting of SEQ ID NO: 1, was conjugated to a cholesterol moiety to form an antagomir. This antagomir was added to the culture media of rat cortical neurons which lead to a significant increase in MeCP2 protein levels (FIG. 7A). This demonstrates that an MRE-concealing LNA can be made cell permeable with the addition of a cholesterol moiety, and the cholesterol modification does not interfere with the activity of the MRE-concealing LNA. The MRE-concealing LNAs delivered in this way provide a means to increase MeCP2 protein expression (FIG. 7B).

In summary, this example (particularly FIGS. 4A, 4B, and 7A) provides evidence that the MRE-concealing LNAs conjugated to a cholesterol moiety can increase the expression of a target gene via interaction at the 3′ UTR.

The following materials and methods are provided to facilitate practice of the present example.

Western blots. Standard methodologies were utilized with anti-MeCP2 (Upstate), anti-CtBP1 (BD Biosciences), or anti-α-tubulin (Sigma) antibodies. Primary antibodies were used in 5% BSA/TBST at a 1:1000 concentration for MeCP2, and CtBP1, or a 1:10,000 concentration for α-tubulin. Secondary HRP-conjugated antibodies were used in 3% milk/TBST at a 1:5000 concentration. The blots were exposed using ECL plus (Amersham).

cDNA constructs, siRNA, oligos, and primers. The miR132 and miR1-1 hairpins were amplified from rat genomic DNA by using the following primers: miR132 forward, 5′-CTAGCCCCGCAGACACTAGC-3′ (SEQ ID NO: 3); miR132 reverse, 5′-CCCCGCCTCCTCTTGCTCTGTA-3′ (SEQ ID NO: 4); miR1-1 forward, 5′-TGGCGAGAGAGTTCCTAGCCTG-3 (SEQ ID NO: 5); miR1-1 reverse, 5′-TGTGCACAACTTCAGCCCATA-3′ (SEQ ID NO: 6). miR132 and miR1-1 were cloned into pCAG. A dicer substrate siRNA against MeCP2 was synthesized by Integrated DNA technologies with the following sequence, 5′-CAUGGAAUCCUGUUGGAGCUGGUCUAC-3′ (SEQ ID NO: 7). The primer sequences for real time PCR are as follows: BDNF I forward, 5′-GGCTGGTGCAGGAAAGCAACAA-3′ (SEQ ID NO: 8), reverse, 5′-CTTGTCAGGCTAGGGCGGGAAG-3′ (SEQ ID NO: 9); BDNF III forward, 5′-CCCAGTCTCTGCCTAGATCAAATGG-3′ (SEQ ID NO: 10), reverse, 5′-ACTCGCACGCCTTCAGTGAGAA-3′ (SEQ ID NO: 11); GAPDH forward, 5-ATCCCAGAGCTGAACGGGAAGC-3′ (SEQ ID NO: 12), reverse, 5′-TTGGGGGTAGGAACACGGAAGG-3′ (SEQ ID NO: 13); 18S forward, 5′-CCGCAGCTAGGAATAATGGA-3′ (SEQ ID NO: 14), reverse, 5′-CCCTCTTAATCATGGCCTCA-3′ (SEQ ID NO: 15). The sequences of the 2′-O-methyl oligoribonucletodies (IDT) are: antisense, GGGCAACCGUGGCUUUCGAUUGUUACUGUGG (SEQ ID NO: 16); scrambled, GGGGACACCUCGGAUUCUUUUGGAUCUGUGGG (SEQ ID NO: 17). The sequence of the LNA oligonucleotides (IDT) are (with modified bases underlined): antisense, 5′-TAACAGTCCTGGTGATATTTGGTCA-3′ (SEQ ID NO: 1); control, 5′-TGTAGACAATAATGTCCATGGCCTT-3′ (SEQ ID NO: 18).

Synthesis of Antagomirs. Antagomirs were synthesized using commercially available monomers according to standard solid phase oligonucleotide synthesis protocols. All oligonucleotides were synthesized by IDT. For antagomirs, i.e., cholesterol conjugated DNA-LNA mixmers, the synthesis was performed commercially by Integrated DNA Technologies (IDT), (Coralville, Iowa).

Cell culture and stimulation. Primary cortical cultures were prepared from P1 rats using standard protocols. Briefly, brains extracted from P1 pups were digested with papain for 1 hour, washed, and triturated by being passed through a 10 mL pipette. Neurons were plated in Neurobasal A supplemented with B-27 (Invitrogen), and 10% FBS. After two hours, the plating media was replaced with Neurobasal A containing B-27. For stimulation experiments neurons 3 DIV were treated with 10 μM forskolin dissolved in DMSO for 6 hours.

Neuronal transfection. P1 cortical neurons were nucleofected (Amaxa) according to the manufacturers protocol. After nucleofection, neurons were cultured for 3-5 DIV. Plasmids were used at a concentration of 3.5 μg per 100 μl of nucleofector solution. Oligos (2′-O-Me and LNA) were used at a concentration of 10 nM. siRNA was used at a concentration of 200 nM.

RT-PCR. RNA was extracted from neurons in culture using the RNeasy kit (QIAGEN). RNA was subjected to DNase (Ambion) treatment and reverse transcribed using SuperScript II and random primers (Invitrogen). PCRs (20 μl) contained 2 μl of 10×PCR buffer, 2.5 mM MgCl₂, 200 μM dNTP (Roche), 0.125 μM primer, 1×SYBR green I (Invitrogen), and 1 unit of Platinum Taq (Invitrogen). PCR was performed on an Opticon OP346 (MJ Research) for 3 min at 94° C. followed by 50 cycles at 94° C. for 15s and 68° for 40s. Each reaction was normalized to 18S.

Statistical analysis. Data with homogenous variances were analyzed by using the two-tailed Student t test. A p-value of <0.001 was considered significant.

### REFERENCES

1. Ainir, R. E., et al. Nut. Genet. 23, 185-1 88 (1999).
2. del Gaudio, D., et al. Genet. Med. 8, 784-792 (2006).
3. Van Esch, H., et al. Am. J. Hurt Genet. 77, 442-453 (2005).
4. Collins, A. L., et al. Hum. Mol. Genet. 13, 2679-2689 (2004).
5. Luikenhuis, S., Giacometti, E., Beard, C. F. & Jaenisch, R. PNAS 101, 6033-6038 (2004).
6. Shahbazian, M. D., Antalffy, B., Armstrong, D. L. & Zoghbi, H. Y. Hum. Mol. Genet. 11, 1 15-124 (2002).
7. Vo, N., et al PNAS 102, 16426-1 643 1 (2005).
8. Leaman, D., et al. Cell 121, 1097-1 108 (2005).
9. I,i, Y., Wang, F., Lee, J. A. & Gao, F. B. Genes Dev. 20, 2793-2805 (2006).
10. Kurreck, J., Wyszko, E., Gillen, C. & Erdmann, V. A. Nucleic acids Res. 30, 191 1-1918 (2002).
11. Wang, H., et al. J Neurosci 26, 1091 1-109 15 (2006).
12. Chang, Q., Khare, G., Dani, V., Nelson, S. & Jaenisch, R. Neuron 49, 341-348 (2006).
13. Fukuda, T., Itoh, M., lchikawa, T., Washiyama, K. & Goto, Y. J. Neuropathol. Exp. Neurol. 64, 537-544 (2005).
14. Matarazzo, V., et al. Mol. Cell. Neuro. 27, 44-58 (2004).
15. Jugloff, D. G., Jung, B. P., Purushotham, D., Logan, R. & Eubanks, J. H. Neurobiol. Dis. 19, 18-27 (2005).
16. Huhlagner G., Simard M J., Mello C C., Zamore P D., 2004. Sequence specific inhibition of small RNA function. PLoS Biology. 2: 0465-0475.
17. Wahlestedt C., et al., 2000. Potent and non-toxic oligonucleotides containing locked Nucleic acids. PNAS. 97: 5633-38.
18. Kurreck J., Wyszko E., Gillen C., Erdmann V A., 2002. Design of antisense oligonucleotides stabilized by locked nucleic acids. Nucleic Acids Research. 30: 1911-1918.
19. Grunweller F., Wyszko E.; Bieber B., Jahnel R., Erdmann V A., Kurreck J., 2003. Comparison of different antisense strategies in mammalian cells using locked nucleic acids, 2′-0-methyl RNA, phosphorothioate and small interfering RNA. Nucleic Acids Research. 3 1: 31 85-3 193.
20. Kauppinen Sakari., Vester B., Wengel J., 2005. Locked nucleic acids (LNA): High affinity targeting of RNA for diagnostics and therapeutics. Drug Discovery Today. 2: 287-290.
21. Krutzfeldt J., Poy M N., Stoffel M., 2006. Strategies to determine the biological function of microRNAs. Nature Genetics. 38: S14-S19.
22. Willard H. F. & Hendrich B. D., 1999. Breaking the silence of Rett Syndrome. Nature. 23: 127-128.
23. Guy J., Gan J., Selfridge J., Cobb S., Bird A, 2007. Reversal of neurological defects in a mouse model of Rett syndrome. Science. 3 15: 11 43-7.
24. Krutzfeldt J. et al., 2005. Silencing of microRNAs in vivo with ‘antagomirs.’ Nature. 438: 685-689.

While certain of the preferred embodiments of the present invention have been described and specifically exemplified above, it is not intended that the invention be limited to such embodiments. It will be apparent to one skilled in the art that various changes and modifications can be made therein without departing from the spirit and scope of the present invention, as set forth in the following claims.

What is claimed is:

1. A method of increasing expression of a protein of interest, comprising contacting a cell comprising a gene encoding the protein of interest with an oligonucleotide which has complementarity with a miRNA recognition element (MRE) in a mRNA encoding said protein of interest.

2. The method of claim 1, wherein said oligonucleotide comprises at least one Locked Nucleic Acid (LNA).

3. The method of claim 1, wherein said protein of interest is selected from the group consisting of a tumor suppressor, an interferon, a cytokine, an antibody, a coagulation factor, or myotropins.

4. The method of claim 1, wherein said cell is present in a patient diagnosed with a disease or disorder which results from under-expression of said protein of interest.

5. The method of claim 1, wherein said oligonucleotide is conjugated to a lipophilic moiety to facilitate entry of said oligonucleotide into said cell.

6. The method of claim 5, wherein the lipophilic moiety is cholesterol.

7. A method for identifying an oligonucleotide which is effective to up-regulate expression of a target gene of interest, comprising;
a) identifying a microRNA recognition element (MRE) in an mRNA encoded by said target gene, and
b) synthesizing a MRE-concealing oligonucleotide, and
c) assessing said oligonucleotide for MRE binding affinity and up regulation of target gene expression.

8. The method of claim 7, wherein the oligonucleotide comprises at least one LNA.

9. The method of claim 7, wherein said oligonucleotide is conjugated to cholesterol to facilitate entry of said oligonucleotide into a cell expressing said target gene of interest.

10. The method of claim 7, wherein said MRE-concealing oligonucleotide comprises a sequence which is substantially complementary to about 15 to 30 contiguous nucleotides of a target MRE in said mRNA.

11. An isolated oligonucleotide, comprising a nucleotide sequence sufficiently complementary to a microRNA recognition element (MRE) comprising about 15 to 30 nucleotides, said MRE being present in an mRNA transcript encoded by a target gene of interest.

12. The oligonucleotide of claim 11, further comprising at least one 2′-modified nucleotide.

13. The oligonucleotide of claim 11, wherein cholesterol is conjugated to the molecule to form an antagomir.

14. The oligonucleotide of claim 12, wherein the 2′-modified nucleotide comprises a 2′-O-methyl.

15. The oligonucleotide of claim 11, which comprises at least one Locked Nucleic Acid (LNA).

16. The oligonucleotide of claim 11, wherein said oligonucleotide comprises SEQ ID NO: 1.

17. The oligonucleotide of claim 16, wherein residues 1, 5, 7, 14, 18, and 23 of SEQ ID NO: 1 are Locked Nucleic Acids.

18. A pharmaceutical composition comprising an antagomir of claim 13 in a pharmaceutically acceptable carrier.

19. A method of increasing the amount of MeCP2 protein levels in a cell, comprising contacting the cell with the antagomir of claim 13, said antagomir comprising SEQ ID NO: 1.

20. The method of claim 19, wherein an effective amount of said antagomir is administered to a patient afflicted with Rett syndrome to alleviate symptoms thereof.

21. A kit comprising a) a first component containing a MRE-concealing LNA oligonucleotide, and b) a second component containing saline or a buffer solution adapted for reconstitution of said LNA oligonucleotide, and c) instructional material.
